(19) United States
(12) Patent Application Publication
(10) Pub. No.: US 2026/0083487 A1
(43) Pub. Date: Mar. 26, 2026

(54) GUIDE PLATE FOR THE ANATOMICAL REALIGNMENT OF BONE FRACTURES

(71) Applicants: UNIVERSITA' DEGLI STUDI DI MILANO, Milano MI (IT); FONDAZIONE IRCCS CA' GRANDA OSPEDALE MAGGIORE POLICLINICO, Milano MI (IT); GPI S.P.A., Trento TN (IT)

(72) Inventors: Alessandro BAJ, Milano MI (IT); Fabio MICHELON, Trento TN (IT)

(21) Appl. No.: 19/112,251

(22) PCT Filed: Sep. 18, 2023
(86) PCT No.: PCT/IB2023/059230

§ 371 Date: Mar. 16, 2025

(30) Foreign Application Priority Data
Sep. 19, 2022 (IT) 102022000019188

(51) Int. Cl. A61B 17/80; A61B 17/00; A61B 17/90; A61B 34/10; B33Y 10/00; B33Y 50/00; B33Y 80/00

(52) U.S. Cl. CPC A61B 17/808; A61B 17/8071; A61B 17/90; A61B 34/10; B33Y 50/00; B33Y 80/00; A61B 2017/00526; A61B 2034/105; A61B 2034/108; B33Y 10/00

(57) ABSTRACT

Guide plate for the anatomical realignment of a plurality of fractured bone portions, comprising a back surface configured such as it can be distinctively coupled to the surface anatomy of the area of said plurality of fractured bone portions and characterized by a first plurality of holes placed on a first portion of the guide plate and a second plurality of holes placed on a second portion of the guide plate, wherein the first plurality of holes is misaligned with respect to the second plurality of holes.

The present invention concerns a process and apparatus for producing a guide plate for the anatomical realignment of bone fractures.

### STATE OF THE ART

As is known, bone fractures can be caused by traumas resulting from different possible scenarios, such as, for example, falls, car accidents, sports activities.

Depending on the severity of the bone trauma suffered, different tools may currently be used to restore the affected fractured bone portions to their mechanical and functional physiological condition.

In some cases, the fractures are such as to have to intervene with surgical operations which provide for the insertion of orthopedic prostheses. In other cases, the use of prostheses which allow to recover anatomical functionality can be provided. Still, in other cases, metal plates, which allow to fix the fractured bone portions that suffered a displacement from their physiological position in the proper position, are used. The level of precision with which the bones are realigned, such as to recover the proper geometric relationship and interaction of the fractured bone portions, is not only crucial for restoring the proper functioning of the affected anatomical areas but also to avoid possible repercussions or secondary problems, which differ depending on the area affected. Such observations are very relevant, in particular in the event of fractures that affect small bones and/or whose precise shape is strictly crucial to their function. Consider, for example, the mandibular bone, which, in addition to having crucial physiological functions related to chewing, phonation and breathing, imparts facial features to the lower part of the face. Other bones/anatomical districts, for which the perfect realignment of the fractured portions is particularly delicate, are those present at the level of the hands and feet and whose incorrect welding could lead to a loss of functionality of the end portion of the limb, thus preventing, for example, being able to perfectly grasp objects or perform fine movements, or walk properly. Vertebral fractures constitute another perfect example of a situation in which it is essential to try to achieve a weld in the most proper position possible, such as to preserve the mechanical functionality of the spinal column, as well as to protect the nerve bundles running through it.

In the cases just described, as well as in anatomical districts characterized by the same problems, it is thus crucial to use, in the event of fractures, a method for realigning the fractured bone portions which allows a high degree of precision in making different bone portions fit to one another again and which is as least operator-dependent as possible, thus ensuring reproducibility and reliability of the operations to be performed.

Generally, for fractured bone portions for which realignment with a high degree of precision is necessary, for example in the order of a millimeter, the reduction of fractures currently occurs by means of a pre-surgical analysis of the affected portions which provides for the acquisition of images, for example by means of computed axial tomography or magnetic resonance imaging. Such images can then be processed by a computer through specific software, such as to generate three-dimensional models of the bone portions. Starting from such models, it is possible to virtually replicate the condition of the bone portions in the condition of trauma (misaligned/dislocated) and to thus simulate the best possible arrangement of the realigned bone structure, thus identifying which movements the surgeon must perform to be able to restore alignment between the different bone fragments. An example of such method is described in U.S. Pat. No. 9,980,763.

In other words, such simulations thus allow the operator to have an indication, albeit virtual, of how to intervene manually on the bone portions. Once the actions aimed at aligning the fractured bone portions have been virtually reconstructed, the real alignment is carried out manually by the surgeon while operating, also with the aid of tie rods and elastic bands which allow to stabilize the bone portions in their final aligned configuration. Once the best possible arrangement has been reconstructed in reality, standard metal fastening plates, available on the market, are used to permanently block the different bone portions to one another.

In case of particular needs, the surgeon can physically intervene on the arrangement of said fastening plates but only in an extremely limited way, by adapting them, for example by partially changing their shape, bending them to make them more similar to the alignment configuration of the specific fracture. Said plates remain however standard manufactured products on which the surgeon has very little room for making changes/adapting to the different cases he/she is faced with in reality.

The known art just described has some drawbacks in terms of reliability of the method, repeatability of operations and guarantee of achieving the precise alignment desired, especially in cases for which it is necessary to have precision in the order of a millimeter. First of all, the manual realignment of the bone portions, which follows what is planned by the pre-surgical simulation, will hardly match exactly what was virtually reconstructed. A second drawback is related to the use of standard fastening plates which, as previously mentioned and despite minor changes the surgeon was able to introduce in the operating room, will never be able to be adapted in a precise and univocal way to the specific realignment of the bone portions. In particular, realignment of the bone portions means both their return in a position in which the surface profiles match and their mutual position in a spatial three-dimensional structure corresponding to the configuration prior to the trauma. This second drawback can adversely affect not only the effective realignment of the affected bone portions but also the time necessary to complete said realignment, thus slowing down the operation as a whole.

In the art, some metal plates customized to the individual patient and obtained with rapid prototyping techniques are only known in the orthodontic field. For example, WO2021088429 describes a special screw to be used in the orthodontic field for dental implants accompanied by a hooking plate especially studied for this application. CN110946663 describes a guide plate achieved by 3D printing, also for orthodontic applications in this specific case and which guide plate allows greater accuracy in locating the tooth implant site, thanks to its structure designed on the tooth and gum surface of the patient. In all cases, these plates are plates used to identify the implantation point of the micro-screw of the tooth to be replaced.

In the field of surgical maxillofacial operations, US2015/0272598 describes plates for realignment of bone portions usable for cases of osteotomies, i.e. surgical operations in which one or more cuts of bone portions are predefined and carried out for rearranging bone portions according to the usual and proper anatomy, followed by the fastening of plates known in the art for fixing the bone portions in the realigned configuration.

Such technique thus allows to realign or displace bone portions to different spatial positions, wherein a pre-surgical planning of the method with which to perform one or more cuts is carried out and the bone portions are subsequently aligned as desired, according to the proper anatomy by means of one or more plates.

Today, there is still a need to provide a process and apparatus for the reduction of bone fractures which can result from traumas such as falls, impacts with obstacles and the like. In these cases, bone fractures are variable and unpredictable in terms of the number of fractured bone portions, thus a bone fracture profile that cannot be compared to scenarios in which the bone portions are separated or cut according to a planned pre-surgical strategy.

Today, there is still the need for a process and apparatus for the reduction of fractures which can overcome the technical problems present in the state of the art just described, and in particular for fractures involving bone portions for which a realignment with a high degree of precision is necessary, for example in the order of a millimeter.

### OBJECTS OF THE INVENTION

Objects of the present invention are to provide a process and system for producing a guide plate for anatomical realignment of a plurality of fractured bone portions, which allows to ensure precise alignment of said fractured bone portions and which is able to ensure high reliability and reproducibility of rejoining and reconstructing process of said bone portions.

Further object of the present invention is to provide a guide plate and a permanent plate which are useful for the anatomical realignment operations of a plurality of fractured bone portions. These and other objects are achieved by the present invention by means of a process and system according to the accompanying independent claims. Preferred aspects are set forth in the dependent claims.

### DESCRIPTION OF THE INVENTION

According to an aspect of the present invention, the process for producing a guide plate for the anatomical realignment of a plurality of fractured bone portions deriving from a bone fracture comprises the steps of:
a) processing a plurality of preemptively acquired images of the fractured bone portions, such as to create a digital three-dimensional reconstruction of said fractured bone portions, which are characterized by a misaligned configuration of said fractured bone portions;
b) processing the images of the fractured bone portions in order to create a digital three-dimensional reconstruction of said bone portions in an aligned configuration;
c) identifying, by means of the three-dimensional reconstruction of the bone portions in an aligned configuration referred to at point (b), a plurality of aligned fastening points at which the bone portions must be mechanically constrained to a permanent plate;
d) constructing, by means of a computer, the three-dimensional geometry of a guide plate, in the misaligned configuration of the fractured bone portions as described at point (a), which is configured such as the back surface of the guide plate is at the surface anatomy at the level of the fracture, i.e. of the area involving the fractured bone portions;
e) constructing, on the three-dimensional geometry of the guide plate referred to at point (d), a first plurality of holes located on a first portion of the guide plate, and a second plurality of holes on a second portion of the guide plate, said holes on the first and second portions of the guide plate matching the aligned fastening points identified at point (c), in said at least two bone portions;
f) constructing, by means of a computer, the three-dimensional geometry of the permanent plate configured such as the back surface of the permanent plate can be distinctively coupled to the surface anatomy of the at least two aligned bone portions, said permanent plate comprising holes at the fastening points, identified at point (c), for said at least two bone portions in the aligned configuration;
g) printing the guide plate obtained at points (d) and (e), and the permanent plate obtained at point (f), by using 3D printing methods.

In the present document “hole” and “fastening point” mean a through hole when referred to the plates. The term “plurality” denotes one or more, preferably more than one.

The term “aligned”, referring to the plurality of holes, intends to denote they are all on the same straight line lying on a single axis. The alignment of the two or more fractured bone portions instead refers to the restoration of the physiological position of the different portions. In the present document, when referring to an alignment or misalignment of the fractured bone portions and holes, reference is always made to a configuration envisioned in a three-dimensional space.

The present invention thus allows to overcome the drawbacks present in the state of the art thanks to the process described and which, in other words, provides for a first step during which various images of the fracture (or fractures) are acquired according to one or more of the numerous possible techniques known to the skilled in the art, such as to be able to reconstruct, thanks to the use of appropriate software, a first virtual three-dimensional image which reproduces the fractured bone portions in their real configuration.

Subsequently, said first three-dimensional image, which reproduces the fractured bone portions in their real configuration (in which the various bone portions are misaligned/dislocated), is virtually reworked such as to obtain a second three-dimensional image in which the different fractured bone portions were brought back to their original position, with the proper alignment and “interlocking” with each another. The proper alignment and “interlocking” between the different fractured bone portions is reproduced by also taking into account the processing of the subjective morphological characteristics of the fractured area as a whole, through images and/or measurements possibly acquired together and/or in parallel with the acquisition of the images of said fractured bone portions.

Fastening points, which will be used to fix the permanent plate, are virtually identified on this second three-dimensional image representing the bone portions in their aligned configuration, so as to make the coupling between the various bone portions and their stabilization as perfect as possible. Said fastening points must at least be two, preferably more than two, for each fractured bone portion.

Once the fastening points have been identified on the second three-dimensional image (i.e. the representation of the aligned bone portions), said fastening points are translated on the first three-dimensional image (i.e. the representation of the misaligned bone portions) by means of the image processing and construction software. Once translated onto the first three-dimensional image, said previously identified points will be misaligned with each other by one “degree of misalignment” which will depend on the misalignment between the different fractured bone portions.

Thus, the design of a guide plate can be made starting from the three-dimensional shape of the fractured bone portions, perfectly matching and adhering thereto and provided with holes in misaligned position (like positions translated from the second to the first image). Said holes in a misaligned position will correspond to fastening points, once the fracture has been reduced and the bone portions have been aligned in the aligned and permanent configuration.

In other words, unlike the known art, in which surgical operations in which one or more cuts of bone portions are predefined and carried out for rearranging the bone portions according to the usual and proper anatomy, the guide plate, thanks to its geometric and volumetric shape, will allow to relocate the fractured fragments to the proper anatomic position (bone fracture reduction) without having to perform surgical cuts on the fractured bone portions.

The design of the permanent plate can be done in the same way, starting from the second three-dimensional image comprising the aligned fastening points.

The designs of the guide plate and permanent plate will then be used for producing, according to 3D printing techniques, two rigid objects perfectly matching in shape with the fractured bone portions in a misaligned (guide plate) and aligned (permanent plate) position.

The fracture reduction and realignment process will thus pass in the hands of the surgeon, who will proceed to lay the printed 3D guide plate provided with misaligned holes on the real misaligned fractured bone portions of the patient to which the plate will be perfectly coupled, thanks to its specific shape designed according to the images of the real fracture. This way, it will be possible to identify the points at which the holes into which the screws are inserted to firmly fix and immobilize the permanent plate in a univocal position directly on the fractured bone portions.

Once said points have been identified and the corresponding holes have been made on the bone portions, the surgeon will proceed to: remove the guide plate, manually align the fractured bone portions with each other such as to bring said holes, identified thanks to the guide plate, into alignment as far as possible, lay the printed 3D permanent plate provided with aligned fastening points on the real aligned fractured bone portions of the patient, start to fix the permanent plate such as to achieve a further improved alignment and permanently fix said plate through screws by using the fastening points.

Said procedure will allow a perfect coupling between the different fractured bone portions and a millimetric alignment of the different parts of the fracture, thanks to the fact that the permanent plate is made of rigid material and was designed by the software and produced with 3D printing techniques, to have a shape better matching that of the bone portions of individual patients in their best realigned configuration, according to the three-dimensional reconstruction created by the software in the second three-dimensional image. The screws, which will be inserted into the fastening points preemptively identified thanks to the misaligned holes present on the guide plate, can be tightened little by little by proceeding according to a cross-like geometry (i.e. by tightening the screws two by two in positions opposite to each other). This further expedient, known to the skilled in the art, will allow to make the advantage determined by the perfect realignment of the bone portions even more effective, so that they match both along the surface profile of the fractured bone portions and along the two surfaces of the bone thicknesses of the bone portions. Moreover, such detail allows to make the advantage determined by the perfect matching between the back surface of the permanent plate and the surfaces of the fractured portions even more effective.

The coupling and repositioning of said fractured bone portions will thus be very accurate and able to reproduce, in a particularly faithful way and also to maintain over time, the second three-dimensional image which reconstructed the fracture according to the best possible arrangement.

According to the present invention, the images of the fractured bone portions are acquired from various angles, such as to be able to obtain the geometric and volumetric arrangement of the fractured bone portions. According to a preferred aspect of the invention, such images are obtained by means of known anatomical imaging techniques, such as for example computerized axial tomography.

First, step (a) allows to process the previously acquired images of the fractured bone portions, which images replicate the condition of real misalignment of said fractured bone portions.

Step (b) allows to process the images of the fractured bone portions, as previously acquired, in order to simulate the aligned configuration of the fractured bone portions as much as possible. In this step, it is thus possible to digitally identify and reconstruct the configuration of the bone portions before the bone fracture occurred and to identify the best alignment configuration of said fractured bone portions such as to restore the original geometry before the bone fracture occurred.

Typically, in step (a) and step (b), the processing of the previously acquired images of the fractured bone portions occurs by processing DICOM (Digital Imaging and Communications in Medicine) images which can be imported into a processor which allows to convert spatial and geometric information of the fractured bone portions into a virtual three-dimensional representation of the fractured bone portions. Typically, the virtual three-dimensional representations of the fractured bone portions can be converted into files with stl, stp, step extension or into other types of files known in the art, such as to be able to allow the processing of the virtual representation of the fractured bone portions.

According to what has been previously described, fastening points, also aligned and which will allow to immobilize the bone portions constituting the fracture in the proper position, are identified in step (c) by means of the three-dimensional reconstruction of the bone portions in an aligned configuration referred to at point (b). Such fastening points can also be translated onto the bone portions in a misaligned configuration referred to at point (a), thus identifying the points in which said bone portions should be pierced.

Advantageously, during step (c), the identification of such fastening points by means of the three-dimensional reconstruction of the bone portions in an aligned configuration allows to strategically select the fastening points that are easiest to access with surgical tools. Alternatively, said selection can be made such as to prevent said fastening points from being in positions such as to interfere, for example, with the position of the nerves present in proximity of the fractured bone portions or, finally, from being in positions that would not be effective for the alignment of the fractured bone portions.

In step (d), it is possible to construct the geometry of a guide plate, in the configuration in which the bone portions are misaligned, as described at point (a).

Such guide plate is characterized by a back surface placed at the surface anatomy of the area of the fracture (i.e. of the region comprising the fractured bone portions).

In other words, such back surface of the guide plate is designed such as the guide plate can be positioned on the plurality of fractured bone portions in a univocal way, thanks to the shape of the back surface of the guide plate. In fact, said back surface is characterized by protrusions and depressions which can extend in three spatial dimensions on coplanar or non-coplanar planes and which coincide respectively with depressions and protrusions in the three spatial dimensions present on the anatomical surface of the respective fractured bone portions, so as to prevent undesired displacements of the guide plate when placed in contact with the fractured bone portions.

In step (e), it is possible to construct a first plurality of holes on a first portion of the guide plate and a second plurality of holes on a second portion of the guide plate on the three-dimensional geometry of the guide plate referred to at point (d). The position of such holes corresponds to the desired fastening points in the bone portions in the aligned configuration.

In other words, on the three-dimensional reconstruction of the misaligned bone portions, thanks to the guide plate the first and second plurality of holes will identify the points in which said bone portions must be pierced, such as, once the manually alignment of the bone portions has been carried out, the holes made therein are aligned, thus allowing to mechanically constrain the fractured bone portions in an aligned configuration by means of the permanent plate.

Advantageously, during steps (d)-(e), the guide plate is designed such as to comprise a back support surface which is distinctively laid down to the geometric configuration of the bone portions constituting the two sides of the fracture and comprises, as previously described, holes that act as guides to be able to identify the points in which the fastening points must be positioned for future hooking of the permanent plate on the bone portions still in their misaligned configuration.

According to an aspect of the invention, during step (f) is carried out the construction of the permanent plate having a plurality of aligned fastening points and configured such as the back surface of said permanent plate can be distinctively coupled to the aligned bone portions.

In fact, once the holes have been made on the misaligned bone portions of the fracture, thanks to the guide plate constructed in steps (d)-(e), the position of the fractured bone portions can be corrected manually such as to align the holes and allow the permanent fastening through the permanent plate constructed in step (f).

In other words, the fastening points in the bone portions, which are misaligned prior to surgical operation, will be aligned at the end of the surgical operation.

Advantageously, the constraint of the permanent plate to the aligned bone portions allows to minimize possible positioning errors which could be visually imperceptible after the manual alignment of the fractured bone portions has been completed.

Step (g) of printing the guide plate and the permanent plate can be, for example, carried out by means of known 3D printing techniques of metal or non metal materials, such as for example but without limitations, steel, a material which has a high degree of mechanical stiffness, good ductility and good resistance against corrosion. Alternatively, another metal material printable by 3D printing is titanium, which is characterized by a high degree of mechanical stiffness, a low specific gravity and good resistance against corrosion, in addition to ensuring a good degree of osseointegration when placed in proximity of bone tissue.

Alternatively, the printing of the guide plate can be carried out by means of known 3D printing techniques of plastic materials, such as for example but without limitations, polyamide.

According to another aspect of the present invention, the manufacturing of the permanent plate can occur with 3D printing techniques using resorbable materials, i.e. materials that chemically degrade over time once the physiological structure of the fractured pre-trauma has been restored. Examples of resorbable materials are, but without limitations, polylactide and its derivatives.

According to a possible aspect of the present invention, the fractured bone portions belong to the zygomaticomaxillary complex and/or the mandible and/or the skull bones.

The areas of the zygomaticomaxillary complex and mandible are areas in which the presence of possible fractures can cause damage which can have an effect on the chewing, breathing and speaking ability of an individual who suffers them. It is thus essential that the alignment of the fractured bone portions is carried out with an accuracy of no more than one millimeter. Advantageously, the guide plate and the permanent plate prepared according to the present invention allow to be distinctively positioned on the anatomical surface of the fractured bone portions under examination, in particular according to a particular aspect of the invention, on the bone portions of the zygomaticomaxillary complex or mandible, and are able to provide precise and reproducible guide for the identification of the points in which the fixing of the permanent plate will occur, as well as the possibility to lock the portions realigned in the best coupling position.

According to a possible aspect, in step (g), the 3D printing method is a direct laser sintering 3D printing. Advantageously, this type of printing allows to obtain a back support surface of the guide plate and permanent plate such as to ensure univocal positioning in the three-spatial dimensions on the anatomical surface of the fractured bone portions in their misaligned and aligned configuration, respectively. Other 3D printing methods known in the art and usable for manufacturing the guide and permanent plates according to the present invention are selective laser melting (SLM), electron beam melting (EBM), binder jetting, multi jet fusion (MJF), fused deposition modeling (FDM), fusion filament fabrication (FFF). The permanent plate can also be produced by means of computerized numerical control (CNC) machines.

A further aspect of the present invention is constituted by a system for carrying out a process from step (a) to step (g) according to what is described above. Such system comprises an image acquisition element for acquiring images of a plurality of fractured bone portions. Typically, the acquisition of images of fractured bone portions occurs by means of magnetic resonance imaging or by means of computerized axial tomography, preferably by means of computerized axial tomography.

The system further comprises a computer for the three-dimensional computer reconstruction of the plurality of fractured bone portions and to allow the design of a guide plate and a permanent plate. As previously described, according to the present invention, the guide plate comprises a plurality of misaligned holes and has a back surface that can be distinctively coupled to the surface anatomy of the plurality of fractured bone portions. The permanent plate also comprises a back surface that can be distinctively coupled to the fractured bone portions in their aligned configuration and a plurality of aligned fastening points for constraining such bone portions in their permanent configuration. Typically, a computer for the three-dimensional reconstruction of images comprises a processor and software which allow to process DICOM files obtainable by means of computerized axial tomography. Such images are imported into the software such as to be able to process DICOM files in order to recreate the three-dimensional representation of the fractured bone portions, identify the nerve bundles, dental roots and any other structures of interest. An example of software used for the three-dimensional reconstruction is Mimics (Materialise BV) software usable, for example, on a computer with a processor adapted to reprocess images.

The system according to the invention further comprises a 3D printing element for printing guide plate and the permanent plate elements. Typically, a 3D printing element is a 3D printer capable of printing with a high degree of accuracy, such as for example with a degree of accuracy between 0.3 mm and 0.5 mm, preferably 0.3 mm.

A further object of the present invention is represented by a guide plate for the anatomical realignment of a plurality of fractured bone portions, obtainable with a process as previously described and comprising a back surface configured such as to be able to be distinctively coupled to the surface anatomy of the area of said plurality of fractured bone portions. Said guide plate is characterized by a first plurality of holes placed on a first portion of the guide plate, and a second plurality of holes placed on a second portion of the guide plate, wherein the first plurality of holes is misaligned with respect to the second plurality of holes and said portions may be coplanar or non-coplanar.

According to a possible aspect, the guide plate is obtained by 3D printing of appropriate materials, for example metal materials, for example but without limitations, steel or titanium.

According to a possible aspect, the guide plate comprises a first plurality of holes misaligned with respect to the second plurality of holes by a distance H between 2 mm and 20 mm, preferably between 2 mm and 7 mm.

However, embodiments of the guide plate, in which the distance H can appropriately be selected according to the type of bone portions involved in the trauma and realignment desired in the specific case under examination, are not to be excluded.

Further object of the present invention is constituted by a permanent plate comprising a back surface of the plate that can be distinctively coupled to the surface of the plurality of aligned boned portions and a plurality of aligned fastening holes.

According to a possible aspect, the permanent plate is obtained by 3D printing of appropriate materials, for example materials, for example but without limitations, steel and titanium.

According to a possible aspect, the guide plate and the permanent plate have a uniform thickness.

Advantageously, said uniform thickness allows, in the case of the guide plate, to make it devoid of points weaker than others and, consequently, sufficiently mechanically robust whenever mechanical stresses are applied during its positioning in proximity of the fracture and during the making of the holes in the fractured bone portions in the misaligned configuration.

In the case of the permanent plate, the uniform thickness allows to have mechanical strength such as to avoid possible breakage in the step in which such plate is mechanically constrained by means of screws to the bone portions in the aligned configuration.

Another object of the present invention is constituted by a system comprising the guide plate and the permanent plate for the anatomical realignment of a plurality of fractured bone portions.

Hereinafter, referring to the appended figures, exemplary and non-limiting embodiments of the present invention will be described, in which:

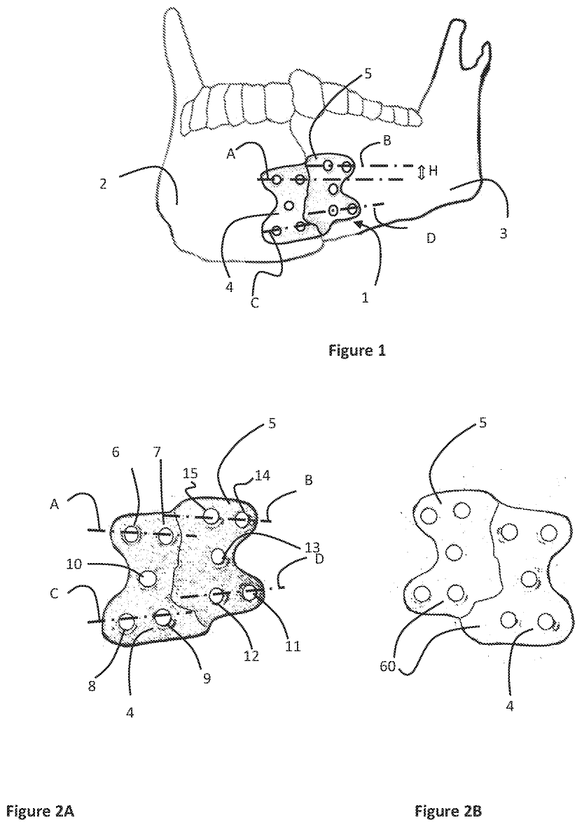

FIG. 1 is a perspective view of the guide plate distinctively positioned on the anatomical surface of a plurality of misaligned fractured bone portions;

FIG. 2A is a front view of the guide plate of FIG. 1;

FIG. 2B is a back view of the guide plate of FIG. 2A;

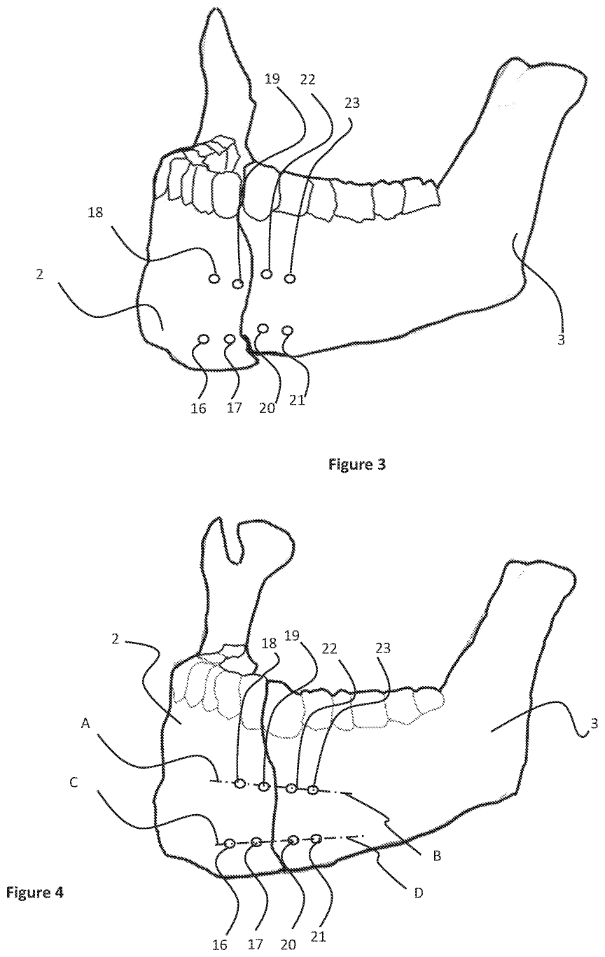

FIG. 3 is a perspective view of the plurality of misaligned bone portions, comprising misaligned fastening holes formed according to the guide plate of FIG. 1;

FIG. 4 is a perspective view of the plurality of realigned bone portions, comprising the holes formed according to the guide plate of FIG. 1 so that said holes are aligned;

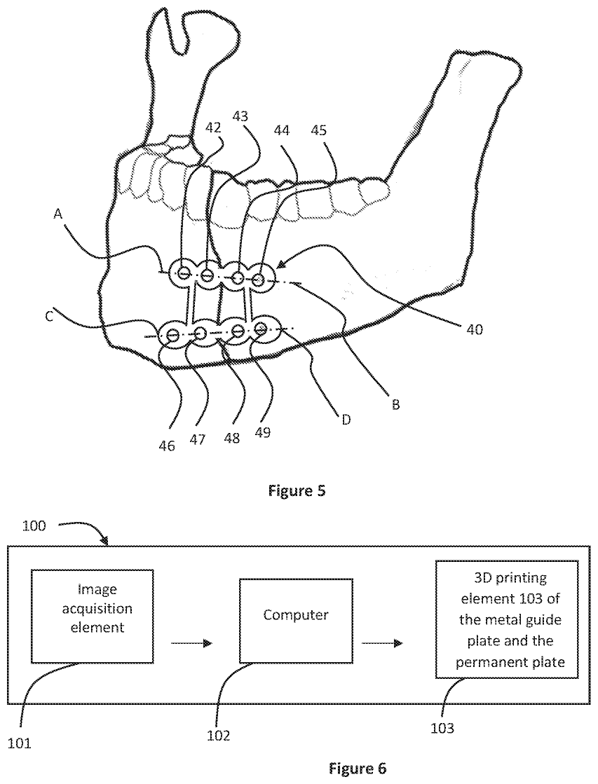

FIG. 5 is a perspective view of the plurality of realigned bone portions of FIG. 4, comprising a schematic representation of the permanent plate with the aligned and concentric fastening holes which allow to mechanically constrain said aligned bone portions;

FIG. 6 is a schematic representation of the system for producing a guide plate.

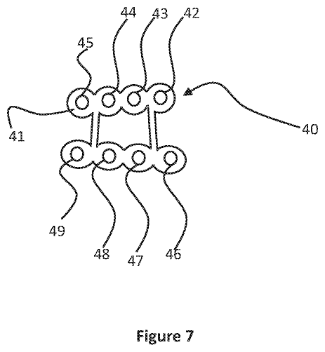

FIG. 7 is a schematic representation of the back surface of the permanent plate of FIG. 5.

A system 100 for producing a guide plate 1 comprises an image acquisition element 101 for acquiring images of a plurality of fractured bone portions 2,3. Typically, said image acquisition element 101 allows to acquire a plurality of images from different angles relative to the central fulcrum of the plurality of fractured bone portions, such as to obtain details of the geometric characteristics as similar as possible to actual anatomy of interest. The system 100 further comprises a computer 102 for the three-dimensional computer reconstruction of the plurality of fractured bone portions 2,3 of the guide plate 1 and of the permanent plate 40. Such guide plate 1, as shown for example in FIG. 2A, comprises a plurality of holes 6-15 and a back surface 60 that can be distinctively coupled to the surface anatomy of the plurality of bone portions 2,3 fractured as shown in FIG. 2B. A permanent plate 40 comprises a plurality of fastening holes 41-49 instead, as better described later.

The system 100 further comprises a 3D printing element 103 for printing the guide plate 1 and the permanent plate 40.

Advantageously, a 3D printing element allows to manufacture a guide plate 1 and a permanent plate 40 based on the specific anatomy of the fracture and being able to reproduce what has been processed by means of an image processing element 101 and a computer 102 for the three-dimensional computer reconstruction of the fractured bone portions 2,3.

The invention further comprises a guide plate 1 for anatomical realignment of a plurality of fractured bone portions 2,3, comprising a back surface 60 configured such as to be distinctively coupled to the surface anatomy of the area of said plurality of fractured bone portions 2,3, which is covered by said guide plate 1. Said guide plate 1 is further characterized by a first plurality of holes 6-10 placed on a first portion 4 of the guide plate 1, and a second plurality of holes 11-15 placed on a second portion 5 of the guide plate 1, wherein the first plurality of holes 6-10 is misaligned with respect to the second plurality of holes 11-15 and said portions 4,5 are non-coplanar. An example of said guide plate is shown, for example, in FIG. 2A (front view) and FIG. 2B (back view).

The guide plate 1 can be positioned on the plurality of fractured bone portions 2,3, thanks to the shape of the back surface 60 of the guide plate 1 since said back surface 60 is characterized by protrusions and depressions in the three spatial dimensions coinciding, respectively, with depressions and protrusions present on the surface of the respective fractured bone portions 2,3, such as to prevent undesired displacements of the guide plate 1 when in contact with the fractured bone portions 2,3.

The guide plate 1 has holes 6-10, in a first portion of the guide plate, that are not aligned with the holes in the second portion 11-15 of the guide plate. In other words, in proximity of the guide plate 1, the axis A passing through the center of the holes 6-7 does not intersect the axis B passing through the center of the holes 14-15 and the axis C passing through the center of the holes 8-9 does not intersect the axis D passing through the center of the holes 11-12, as shown, for example, in FIG. 2A.

Advantageously, in use, the holes of the first portion 6-10 and the second portion 11-15 of the guide plate 1 have a guiding functionality such as to be able to obtain the holes 16-23 in the respective fractured bone portions 2,3 by means of tools known in the art, such as high precision metal pre-implant metal burs, drills provided with drill bits.

Advantageously, in use, once the manual operation for realigning the fractured bone portions has been completed, the holes 16-23 of the fractured bone portions are substantially aligned as shown, for example, in FIG. 4. In other words, the axis A passing through the holes 18, 19 coincides with the axis B passing through the holes 22,23 and the axis C passing through the holes 16,17 coincides with the axis D passing through the holes 20,21.

Advantageously, the central holes 10, 13 of the guide plate 1, which are positioned in a substantially central position in the respective first and second portion, allow to firmly hold the guide plate 1 in position with respect to the anatomical surface of the fractured bone portions, during the step of drilling the fractured bone portions. Such operation can, for example, be carried out by means of appropriate constraining elements, such as screws or other interlocking elements known to the technician of the field.

According to a possible aspect, the guide plate 1 is made of steel or titanium and is obtained by 3D printing.

Advantageously, the guide plate 1 made of steel or titanium and obtained by 3D printing allows to have a first portion of the guide plate 1 and a second portion 5 of the guide plate 1 in a non-coplanar arrangement adapted to ensure the adhesion of the guide plate 1 to the fractured bone portions which can be characterized by complex anatomy. In other words, the adhesion of the guide plate 1 to the anatomy of the fractured bone portions 2,3 can be ensured on a plurality of planes shared by the guide plate 1 and the anatomical surface of the fractured bone portions.

According to a possible aspect, the guide plate 1 comprises a first plurality of holes 6-10 misaligned with respect to the second plurality of holes 11-15 by a distance H between 2 mm and 20 mm, preferably between 2 mm and 7 mm.

Such a distance H is defined as the distance in a direction substantially perpendicular to the main extent direction of the axes A and B and/or to the main extent direction of the axes C and D, as shown, for example, in FIG. 1.

According to a possible aspect, a permanent plate 40 comprises a back surface 41 of the plate that can be distinctively coupled to the surface of the plurality of the aligned bone portions and comprising a plurality of fastening holes 42-49 aligned and concentric to a plurality of holes 16-23 in the fractured bone portions 2,3 in the aligned configuration, as shown, for example, in FIG. 5.

The back surface 41 of the permanent plate 40 is the surface in contact with the anatomical surface of the fractured bone portions 2,3 in the aligned configuration.

More in detail, the aligned bone portions can be mechanically constrained to one another by means of the permanent plate 40 provided with fastening holes 42-49 corresponding to the holes 16-23 in the respective fractured bone portions 2,3 and manually aligned.

According to a possible embodiment, a permanent plate 40 can have a main extent length substantially parallel to the axis A and the axis B or the axes C and D and comprising fastening points 42,43,44,45 corresponding to the holes 18,19,22,23, and fastening points 46,47,48,49 corresponding to the holes 16,17,20,21.

The permanent plate 40 is mechanically constrained to the bone portions 2,3 aligned by means of constraining elements such as for example screws and by gradual tightening until the bone portions 2,3 are aligned according as planned in the pre-surgical step and such as to ensure minimization of alignment error which may not be visually perceivable. Preferably, the gradual tightening occurs by means of a cross-like tightening, i.e. by tightening a screw in the hole 46 of the permanent plate 40 and in the hole 16 of the aligned boned portion 2, followed by tightening a screw in the hole 45 of the guide plate 40 and in the hole 23 of the aligned bone portion 3, followed by tightening a screw in the hole 42 of the guide plate 40 and in the hole 18 of the bone portion 2, followed by tightening a screw in the hole 49 of the guide plate 40 and in the hole 21 of the bone portion 3.

According to a preferred aspect of the invention, the permanent plate 40 is made of steel or titanium and is obtained by 3D printing.

According to a possible aspect, the guide plate 1 or the permanent plate 40 have a uniform thickness.

The invention further comprises a system comprising the guide plate 1 and the permanent plate 40 for the anatomical realignment of a plurality of fractured bone portions 2,3.

A further aspect of the present invention concerns a process for producing a guide plate 1 for the anatomical realignment of a plurality of fractured bone portions 2,3 deriving from a bone fracture, comprising the steps of: a) processing a plurality of preemptively acquired images of the fractured bone portions 2,3, such as to create a digital three-dimensional reconstruction of said fractured bone portions 2,3, which are characterized by a misaligned configuration of said fractured bone portions 2,3 characterized by a misaligned configuration of said fractured bone portions; b) processing the images of the fractured bone portions 2,3 such as to create a digital three-dimensional reconstruction of said bone portions 2,3 in an aligned configuration; c) identifying, by means of the three-dimensional reconstruction of the bone portions in an aligned configuration referred to at point (b), a plurality of aligned fastening points 42-49 in which the bone portions 2,3 must be mechanically constrained to the permanent plate 40; d) constructing, by means of a computer, the three-dimensional geometry of a guide plate 1, in the misaligned configuration of the fractured bone portions 2,3 as described at point (a), configured such as the back surface 60 of the guide plate 1 is at the surface anatomy at the level of the fracture, i.e. of the area involving the fractured bone portions; e) constructing, on the three-dimensional geometry of the guide plate 1 referred to at point (d), a first plurality of holes 6-10 placed on a first portion 4 of the guide plate 1, and a second plurality of holes 11-15 on a second portion of the guide plate 5, said holes on the first and second portions of the guide plate matching the aligned fastening points 42-49 identified at point (c), in said at least two bone portions 2,3; f) constructing, by means of a computer, the three-dimensional geometry of the permanent plate 40 configured such as the back surface 41 of the permanent plate can be distinctively coupled to the surface anatomy of the at least two aligned bone portions 2,3, said permanent plate comprising holes at the fastening points 42-49 identified at point (c), for said at least two bone portions 2,3 in the aligned configuration; g) printing the guide plate 1 obtained at points (d) and (e) and the permanent plate obtained at point (f) by using 3D printing methods.

According to a preferred aspect, the fractured bone portions 2,3 belong to the zygomaticomaxillary complex and/or the mandible and/or the skull bones.

In particular, in the case of the zygomaticomaxillary complex and/or the mandible, realignment and fastening of fractured bone portions 2,3 by using a guide plate 1 and a permanent plate 40 can ensure alignment with a degree of precision in the order of a millimeter, thus capable of meeting the peculiar needs determined by the fracture of this specific bone and therefore able to ensure that, once fixed in the proper position and welded, the subject will be able to have a correct chewing, phonation and breathing.

According to a possible aspect of the invention, in step (g), the 3D printing method for printing the guide plate 1 and the permanent plate 40 is direct laser sintering.

1) A process for producing a guide plate (1) for the anatomical realignment of a plurality of fractured bone portions (2,3), said process comprising the steps of:
a) processing a plurality of preemptively acquired images of the fractured bone portions (2,3) to create a digital three-dimensional reconstruction of said fractured bone portions (2,3), which are characterized by a misaligned configuration of said fractured bone portions (2,3);
b) processing the images of the fractured bone portions (2,3) to create a digital three-dimensional reconstruction of said fractured bone portions (2,3) in an aligned configuration;
c) identifying, by means of the three-dimensional reconstruction of the fractured bone portions in the aligned configuration of step (b), a plurality of aligned fastening points (42-49) at which the fractured bone portions (2,3) are to be mechanically constrained to a permanent plate (40);
d) generating, by means of a computer, a three-dimensional geometry of a guide plate (1), to cooperate with the misaligned configuration of the fractured bone portions (2,3) of step (a), such that a back surface (60) of the guide plate (1) is at the surface anatomy level of the the area involving the fractured bone portions;
e) generating by means of a computer, on the three-dimensional geometry of the guide plate (1), a first plurality of holes (6-10) on a first portion (4) of the guide plate (1) and a second plurality of holes (11-15) on a second portion of the guide plate (5), said first and second plurality of holes corresponding to the aligned fastening points (42-49) in said at least two bone portions (2,3);
f) generating, by means of a computer, a three-dimensional geometry of the permanent plate (40) configured such that a back surface (41) of the permanent plate (40) can be distinctively coupled to the surface anatomy of the fractured bone portions (2,3) in the aligned configuration, said permanent plate comprising holes at the fastening points (42-49) of said at least two bone portions (2,3) in the aligned configuration; and
g) printing the guide plate (1) generated according to steps (d) and (e), and the permanent plate (40) generated according to step (f), using 3D printing methods.

2) The process according to claim 1, wherein the fractured bone portions (2, 3) belong to at least one of a zygomaticomaxillary complex, a mandible and bones of a skull.

3) The process according to claim 1, wherein the 3D printing methods of step (g) comprise direct laser sintering.

4) A system (100) for producing the guide plate (1) according to claim 1, comprising:
an image acquisition element (101) for acquiring images of the plurality of fractured bone portions (2,3);
a computer (102) for three-dimensional computer reconstruction of the plurality of fractured bone portions (2,3), the guide plate (1) and the permanent plate (40); and
a 3D printing element for printing the guide plate (1) and the permanent plate (40).

5) A guide plate (1) for the anatomical realignment of a plurality of fractured bone portions (2,3) obtainable according to the process of claim 1,
the guide plate comprising said back surface (60) configured such that it can be distinctively coupled to the surface anatomy level of the area involving said plurality of fractured bone portions (2,3) is covered by said guide plate (1), said guide plate (1) comprising said first plurality of holes (6-10) on said first portion (4) of the guide plate (1) and said second plurality of holes (11-15) on said second portion (5) of the guide plate (1),
wherein the holes of the first plurality of holes (6-10) are not aligned with the holes of the second plurality of holes (11-15) and said first and second portions (4,5) are not coplanar.

6) The guide plate (1) according to claim 5, wherein said guide plate comprises steel or titanium and is obtained by 3D printing.

7) The guide plate (1) according to claim 5, wherein said guide plate comprises polyamide and is obtained by 3D printing.

8) A permanent plate (40) obtainable according to the process of claim 1, the permanent plate comprising said back surface (41) adapted to be distinctively coupled to the surface anatomy of the plurality of fractured bone portions in the aligned configuration and comprising said plurality of fastening holes (42-49) aligned and concentric to a plurality of holes in the fractured bone portions (2,3) in the aligned configuration.

9) The permanent plate (40) according to claim 8, wherein said permanent plate (40) comprises steel or titanium and is obtained by 3D printing.

10) The guide plate (1) according to claim 5, wherein said first and second portions have a uniform thickness.

11) A system comprising the guide plate (1) and the permanent plate (40) formed according to the process of claim 1 for the anatomical realignment of a plurality of fractured bone portions (2,3),
the guide plate comprising said back surface (60) configured to be distinctively coupled to the surface anatomy level of the area of said involving the plurality of fractured bone portions (2,3), said guide plate (1) comprising said first plurality of holes (6-10) on said first portion (4) and said second plurality of holes (11-15) on said second portion (5),
wherein the first plurality of holes (6-10) are not aligned with the second plurality of holes (11-15) and said first and second portions (4,5) are not coplanar, and
the permanent plate comprising said back surface (41) adapted to be distinctively coupled to the surface anatomy of the plurality of fractured bone portions in the aligned configuration and comprising said plurality of fastening holes (42-49) aligned and concentric to a plurality of holes in the plurality of fractured bone portions (2,3) in the aligned configuration.

12) The permanent plate (40) according to claim 8, wherein said permanent plate has a uniform thickness.

13) The system according to claim 4, wherein the fractured bone portions (2, 3) belong to at least one of a zygomaticomaxillary complex, a mandible and bones of a skull, and wherein the 3D printing methods of step (g) comprise direct laser sintering.

14) A system for the anatomical realignment of a plurality of fractured bone portions (2,3), the system comprising a guide plate (1) and a permanent plate (40),
the guide plate (1) comprising a back surface (60) configured to be distinctively coupled to a surface anatomy level of an area of said plurality of fractured bone portions (2,3) to be covered by said guide plate (1), said guide plate (1) further comprising a first plurality of holes (6-10) on a first portion (4) of the guide plate (1) and a second plurality of holes (11-15) on a second portion (5) of the guide plate (1), and wherein the first plurality of holes (6-10) is not aligned to the second plurality of holes (11-15) and said first and second portions (4,5) are not coplanar, and
the permanent plate (40) comprising a back surface (41) adapted to be distinctively coupled to a surface anatomy of an area of said plurality of fractured bone portions in an aligned configuration and comprising said plurality of fastening holes (42-49) aligned and concentric to a plurality of holes in the fractured bone portions (2,3) in the aligned configuration.
